(19) United States
(12) Patent Application Publication
(10) Pub. No.: US 2002/0146371 A1
(43) Pub. Date: Oct. 10, 2002

(54) Methods for development and use of diagnostic and therapeutic agents

(76) Inventors: King Chuen Li, Bethesda, MD (US); Mark David Bednarski, Los Altos, CA (US)

Correspondence Address: PAMELA J. SHERWOOD, Bozicevic, Field and Francis LLP, Suite 200, 200 Middlefield Road, Menlo Park, CA, 94025, US

(21) Appl. No.: 10/004,348

(22) Filed: Oct. 18, 2001

Related U.S. Application Data
(60) Provisional application No. 60/241,574, filed on Oct. 18, 2000.

(51) Int. Cl. A61K 51/00; A61K 49/04; C12Q 1/68

(52) U.S. Cl. 424/1.73; 424/9.35; 424/9.6; 435/6; 424/9.43

(57) ABSTRACT

In vivo imaging of disease associated tissues, including tumors and other malignant growths, infection and inflammation, is used in the discovery, screening and development of therapeutic and/or diagnostic molecular targets for intervention in the treatment of the diseases involved. In vivo imaging is used to detect spatial and temporal variations in the imaging features of disease associated tissues. The physical regions of the tissue that correlate with imaging features are then assessed for patterns of gene expression. The corresponding genes or gene products that are upregulated in the regions of interest are useful as therapeutic and imaging targets, with enhanced spatial and/or temporal specificity.

The recent explosion of information in the fields of genomics and proteomics has provided a rich ground for the discovery of molecular targets against which therapeutic and/or diagnostic agents can be directed. Tissues for potential target discovery may include tumors and other malignant growths, or infected or inflamed tissues. For example, methods have been described for gene expression profiling of tumor cells (see any one of Ono et al. (2000) Cancer Res. 60(18):5007-11; Svaren et al. (2000) J Biol Chem.; or Forozan et al. (2000) Cancer Res. 60(16):4519-25 for examples). Similarly, proteomics has been used to profile the protein expression in tumor samples (see Minowa et al (2000) Electrophoresis 21(9):1782-6; Cole et al. (2000) Electrophoresis 21(9):1772-81; Simpson et al. (2000) Electrophoresis 21(9): 1707-32); etc.

In addition, functional genomics is a new and powerful tool for assessing large arrays of hundreds or thousand of genes being expressed in a tissue sample. These methods have recently been used to define new ‘taxonomic’ forms of cancers and to follow changes during cancer chemotherapy. However, these current methods of tissue analysis for discovery of new imaging and therapeutic agents do not take into consideration the spatial and temporal variation in gene and protein expression within the target tissues. There is a need to resolve the tissue analysis data both spatially and temporally so that the most relevant targets can be identified. Similarly, there is a clinical need to be able to determine the location and/or extent of sites of focal or localized lesions for initial evaluation, and for following the effects of therapy.

From the time of the first X-ray, in vivo imaging has provided a vital function for medical research and diagnosis, by permitting the clinician to assess, in real time and space, what is happening within the patient's body. In addition to nuclear medicine and MRI, other imaging methods including positron emission tomography (PET), computerized tomography (CT), ultrasonography (US), optical imaging, infrared imaging, in vivo microscopy and x-ray radiography have also been used for obtaining morphologic, metabolic and functional information of living tissues in vivo in a spatially and temporally resolved manner.

For example, magnetic resonance imaging (MRI) is an imaging technique used primarily in medical settings to produce high quality images of the inside of the body. MRI is based on the absorption and emission of energy in the radio frequency range of the electromagnetic spectrum. Although there is a limitation on imaging objects smaller than the wavelength of the energy being used to image, MRI gets around this limitation by producing images based on spatial variations in the phase and frequency of the radio frequency energy being absorbed and emitted by the imaged object.

Contrast enhanced MRI is a powerful tool for the diagnosis of a variety of malignancies. MRI has both high spatial and temporal resolution, with current imaging systems capable of visualizing changes in tissue contrast with micron spatial resolution and millisecond temporal resolution. It has been demonstrated that malignant tumors tend to have faster and higher levels of enhancement when compared to normal surrounding tissues. Furthermore, the kinetics of contrast enhancement on MRI has been correlated to tumor grades and aggressiveness in different tumors. The precise mechanism and origin of contrast enhancement in tumors therefore seems to be related to the complex biological processes associated with tissue perfusion and vascular permeability such as neovascularization and tumor angiogenesis. This may account for the correlation between tumor grade and aggressiveness and contrast enhancement on MRI.

In the field of nuclear medicine, pathological conditions are localized by imaging the internal distribution of administered radioactively labeled tracer compounds that accumulate specifically at the pathological site. A variety of radionuclides are known to be useful for radioimaging, including ⁶⁷Ga, ^{99m}Tc, ¹¹¹In ¹²³I, ¹²⁵I, ¹⁶⁹Yb and ¹⁸⁶Re. In PET, positron emitting isotopes are conjugated to tracer compounds that also accumulate in pathologic tissues.

Specificity of accumulation may be provided by conjugating the radioactive tracer to a binding moiety that binds to the cells of interest. Many examples of such binding moieties have been used experimentally and clinically. For example, anticancer antibodies labeled with different radionuclides have been studied in human tumor xenografts and in clinical trials. Molecular targets for binding moieties include a variety of tumor-associated antigens. For example, in breast cancer, these molecular targets have included carcinoembryonic antigen (CEA) and the polymorphic epithelial mucin antigen, MUC1, and more recently the growth factor receptors, EGF-R and HER-2/neu. Imaging and image-guided therapeutic agents that target the alpha-v-beta-3 integrin have utilized antibodies conjugated to a liposome surface. Such agents can show changes in spatial and temporal distribution of the receptor using imaging.

Alternatively, radiolabelled peptides have been used for imaging a variety of tumors, infection/inflammation and thrombus. A number of ^{99m}Tc-labelled bioactive peptides and peptidomimetics have proven to be useful diagnostic imaging agents. Due to their small size, these molecules exhibit favorable pharmacokinetic characteristics, such as rapid uptake by target tissue and rapid blood clearance, which potentially allows images to be acquired earlier following the administration of ^{99m}Tc-labelled radiopharmaceuticals.

Currently genomic and proteomic analysis is performed on tumor samples without consideration of known differences in imaging patterns within the same tumor over space and time. It is of interest to determine whether the upregulation of gene expression can be correlated with imaging information, thereby allowing imaging to serve as a powerful non-invasive tool for characterizing different regions of tumors and other lesions.

### SUMMARY OF THE INVENTION

Methods and compositions are provided for the discovery, screening and development of novel therapeutic and/or diagnostic targets, based on the use of in vivo imaging of lesions to detect spatial and temporal variations in gene and protein expression. Using this approach, targets for specific processes can be elucidated for the development of therapeutic and diagnostic agents that may have missed without spatial and temporal resolution provided by image guided tissue analysis. Imaging may be performed using agents that enhance tissue or organ images or nuclear spectra obtained with radioisotope scanning; magnetic resonance imaging (MRI), MR spectroscopy (MRS); positron emission tomography (PET); computerized tomography (CT); ultrasonography (US); optical imaging; infrared imaging; in vivo microscopy; or x-ray radiography, and may be used in initial evaluation, to follow the progression of disease, or course of treatment. Variations in the imaging analysis, e.g. the contrast enhancement observed with MRI, etc., are correlated with alterations in gene or protein expression, and used to select optimal targets for therapy and imaging.

In another embodiment of the invention, the imaging information is used to develop and utilize individualized diagnostic and image-guided agents, based on the information provided by detailed analysis of gene expression in tissue samples. The choice of imaging agents is determined by patient sample analysis, which analysis may include hybridization to a polynucleotide array, proteomic analysis, mass spectroscopy, gel electrophoresis, antibody arrays, direct screening of the affected tissue by tissue arrays, and the like. The optimal molecular targets for imaging are determined by this analysis, and one or a cocktail of image-enhancing agents, contrast agents or spectral shift agents are selected. The imaging information can also be used for selection or modification of treatment methods and/or agents at any point of the treatment process.

### BRIEF DESCRIPTION OF THE DRAWINGS

FIG. 1. Patient #1. Coded high-resolution MR image (FIG. 1A) and dynamic contrast enhancement data (FIG. 1B) of the invasive carcinoma in Patient #1. The high-resolution image is color-coded with the K₂₁ values on a pixel-by-pixel basis superimposed on the gray-scale image. There is some variation of the K₂₁ values over the different regions of the tumor.

FIG. 2 demonstrates a time-intensity curves of the inner and peripheral regions of the large tumor.

FIG. 3 demonstrates time intensity curves of a tumor.

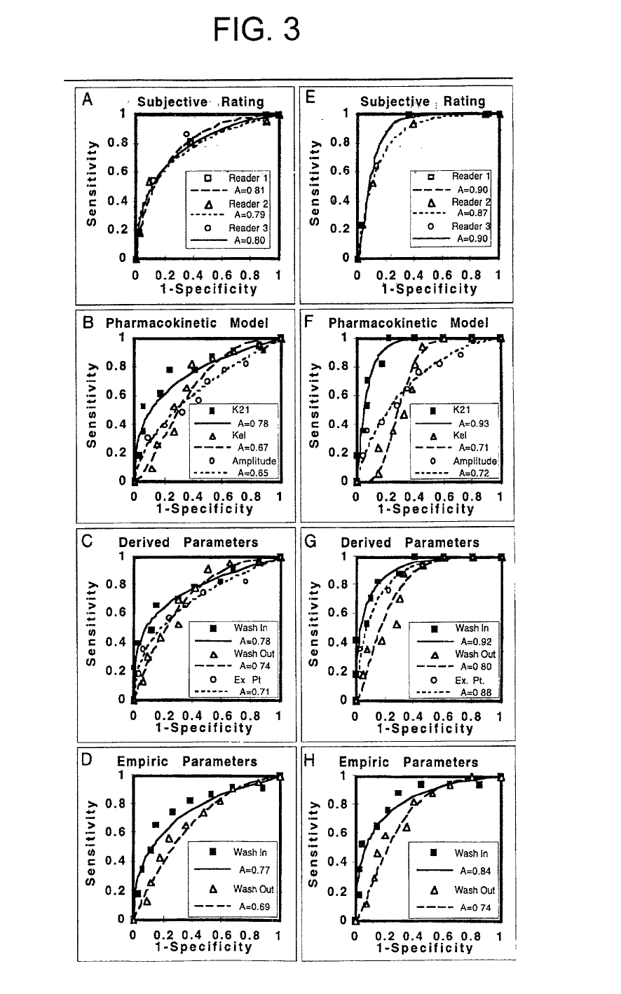

FIGS. 4A, 4B and 4C. MRI of a C3H/K mouse with an implanted subcutaneous squamous cell carcinoma VII tumor (tumor A from table 1). Images obtained on a clinical 3T GE Signa MR scanner (256×192, FOV 8, 4 NEX, slice thickness 1.5 mm, 12 slices/tumor). (A) T2-weighted image (FSE, TR/TE 4000/85, ET=8). No significant enhancement is observed indicating a solid mass with no significant necrosis. (B) Pre-contrast T1-weighted image (SE TR/TE 300/13). No contrast is observed in the tumor. (C) Contrast-enhanced T1-weighted image (Gd(DTPA): Magnevist®, Berlex, Wayne, N.J.; 200 μl of 0.5M solution administered via tail vein). The image was obtained approximately 10 minutes after injection. The thick arrow indicates the contrast-enhanced area, and the thin arrow indicates the non-enhanced area. Tissue was removed from these respective areas for genomic analysis.

FIGS. 5A and 5B. H&E stained tissue sample from the contrast-enhanced area (A), and non-enhanced area (B). Sections of tumor from both enhanced and non-enhanced regions are histologically similar despite the change in the contrast enhanced MRI. Both regions of the tumor are composed of sheets of moderately pleomorphic large cells with eccentrically placed nuclei and abundant eosinophilic cytoplasma with distinct cell borders. There are a few clusters of pyknotic cell debris and scattered throughout the fields of tumor cells are low numbers of fibroblast-like cells and blood vessels. No necrosis is observed at the stage of tumor growth, and no significant inflammatory infiltrates (neutrophils, lymphocytes) are observed.).

FIG. 6 shows a gene expression profile from non-enhanced and enhanced regions of a tumor.

FIG. 7 shows a proteomics profile in contrast enhanced and non-enhanced regions.

Table 1 shows a summary of K₂₁ values and gene expression data in patients 1-3.

### DETAILED DESCRIPTION OF THE EMBODIMENTS

In vivo imaging of disease associated tissues, including tumors and other malignant growths, infection and inflammation, is used in the discovery, screening and development of therapeutic and/or diagnostic targets for intervention in the treatment of the diseases involved. In vivo imaging is used to detect spatial and temporal variations in the features of disease-associated tissues. The physical regions of the tissue that correlate with imaging features are then assessed for patterns of gene and protein expression. The corresponding genes or gene products are useful in the design of therapeutic and/or diagnostic and imaging targets, with enhanced spatial and/or temporal specificity.

The specific imaging techniques selected will vary with the type of disease, tissue, patient status, etc. The methods may include magnetic resonance imaging, radioisotope scanning; NMR imaging; spectroscopy; positron emission tomography (PET); computerized tomography (CT); ultrasonography (US); optical imaging; infrared imaging; and x-ray radiography.

Mammalian species that provide tissue for analysis include canines; felines; equines; bovines; ovines; etc. and primates, particularly humans. Animal models, particularly small mammals, e.g. murine, lagomorpha, etc. may be used for experimental investigations. Animal models of interest include those for models of tumors, immune responsiveness, and the like.

Regions and/or time points of interest for screening are detected by the imaging methods, and are visualized through the methods appropriate to the imaging technique. The tissue regions corresponding to the imaging features of interest are then further analyzed for the presence of variation in mRNA or protein levels, e.g. to correlate the expression patterns with the observed imaging variation. It has been found that imaging, for example using contrast enhanced MRI, provides a powerful tool for characterizing different regions of tumors for genomic and proteomic analysis. Variations in the features of the tissue, e.g. tumor tissue, are found, both spatially and temporally, and can be observed with imaging. These features are found to correlate with changes in gene and protein expression profiles. Identification of the genetic or protein sequence of interest is accomplished using methods known in the art for functional genomics and proteomics.

The targeted gene or gene product may then be used as a target for drug screening, for targeted therapeutic intervention, diagnostic uses, and for further targeted imaging, e.g. to follow the course of disease through drug treatment, and the like.

In one embodiment of the invention, the imaging analysis is used to guide selection of patient appropriate agents for imaging and therapy. A particular advantage of the invention is the ability to provide individualized imaging and therapy, taking advantage of differences in patient specific gene expression and imaging patterns.

Preferred imaging methods provide an enhanced signal at the site of diseased tissue, e.g. tumors, infectious lesions, etc., where the enhancement generally correlates with expression of one or more genes of interest. Such imaging methods may comprise target specific binding moieties. Alternatively non-specific contrast agents may be used, wherein the distribution and the pharmacokinetics of the contrast agent correlates with the presence of one or more genes and proteins of interest, e.g. proteins altering microvascular permeability, etc.

In one embodiment of the invention, molecular targets that are suitable for imaging or therapy are identified based on patterns of gene expression, and antibodies and ligands developed that have high specificity for the targeted tissue. Patient samples are assessed for expression of genes corresponding to such molecular targets, and the selection of agents optimized by analysis of this information. The imaging agent or cocktail of imaging agents is individualized, and used to monitor spatially and temporally the tissue of interest.

The information obtained from image analysis is used to monitor treatment, modify therapeutic regimens, and to further optimize the selection of imaging agents. With this approach, therapeutic and/or diagnostic regimens can be individualized and tailored according to the imaging data obtained at different times over the course of treatment, thereby providing a regimen that is individually appropriate. In addition, image-guided biopsies of tissues can be obtained at any point during the treatment process and the tissue specimens provided for genomics and proteonomics and other analyses. The combination of imaging and image-guided genomics and proteomics can provide information about the target tissues that is both spatially and temporally resolved. In this way targets that may be missed because of sampling inappropriate regions of tissues at inappropriate times, which can be resolved through image guided tissue analysis. Examples are provided demonstrating that major differences in genomics and proteomics occur within the same tumor and that these changes, indicated by imaging changes, can be associated with the extracellular matrix (ECM) genes and proteins and the metastatic potential of the tumor.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a compound” includes a plurality of such compounds and reference to “the agent” includes reference to one or more agents and equivalents thereof known to those skilled in the art, and so forth. All technical and scientific terms used herein have the same meaning as commonly understood to one of ordinary skill in the art to which this invention belongs unless clearly indicated otherwise.

In vivo Imaging: as used herein the term refers to the radiological production of an in vivo image using X-rays, ultrasound, computed tomography, magnetic resonance, radionuclide scanning, optical or thermography. Methods such as ultrasound may rely on the nature of the tissue for the image, while other methods, such as magnetic resonance, radionuclide scanning, optical, etc. may utilize an imaging agent for enhanced contrast. Imaging agents may be targeted ligand specific or non-specific. Imaging features are the regions of enhanced contrast, etc. that provide for the in vivo image.

Ligand specific Imaging Agent: as used herein refers to a binding moiety that specifically binds through chemical or physical means to a target or marker on the surface of a cell to be imaged, and which can be used for imaging or therapy. The imaging agent may comprise a ligand specific for a protein that is upregulated in the targeted tissue, e.g. antibodies specific for tumor associated antigens; may bind to compounds present in the diseased tissue, e.g. LPS in bacterial infections, annexin binding to phosphatidylserine in the membranes of cells undergoing apoptosis; may be concentrated in regions where the vascular permeability is changed; and the like.

The binding portion of the imaging agent is conjugated to an image-enhancing agents, contrast agents or spectral shift agents to enhance tissue or organ images or nuclear spectra obtained with radioisotope scanning or NMR imaging or spectroscopy or positron emission tomography (PET), or computerized tomography (CT), or ultrasonography (US), or optical imaging or x-ray radiography. Alternatively, the imaging agent may be activated to provide contrast by the presence of proteins, such as enzymes, or nucleic acids. For therapeutic purposes, the binding moiety is bound to a pharmacologically active moiety, e.g. toxins, radioisotopes and the like.

Binding moieties include antibodies, lectins that bind to specific carbohydrates of interest; peptide ligands and peptidomimetics that bind to receptors on the cell surface; hormones that bind to their cognate receptor; annexin, and the like. The specific binding pairs may include analogs, derivatives and fragments of the original specific binding member. For example, an antibody directed to a protein antigen may also recognize peptide fragments, chemically synthesized peptidomimetics, labeled protein, derivatized protein, etc. so long as an epitope is present. Recombinant DNA methods or peptide synthesis may be used to produce chimeric, truncated, or single chain analogs of a peptide or polypeptide binding moiety. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well known to those skilled in the art. For imaging enzymes as targets, such proteins as proteases, glycosidases, phosphatases, kinases, etc. are targets that have inhibitors that can serve as catalytically active imaging agents.

Molecular targets of interest for imaging tumors and/or angiogenesis include, but are not limited to, cell surface adhesion molecules, e.g. selectins, adherins, integrins, etc.; cell surface receptors, e.g. those specific for growth factors, hormones, cytokine, chemokines, etc.; ion channels and transporters; etc. Of particular interest are markers that are over-expressed in the target tissue, relative to normal cells, or to surrounding tissue. These markers are also of interest for imaging inflammation, for example in following progress of treatment for bacterial infections, autoimmune diseases, etc.

Potential tumor antigens for immunotherapy include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A; MAGE-1, MAGE-3; GAGE family; etc.; viral antigens, e.g. human papilloma virus, Epstein Barr virus etc.; tissue specific self-antigens, e.g. prostate specific antigen, prostate specific membrane antigen; thyroglobulin, α-fetoprotein; etc.; and over-expressed self antigens, e.g. her-2/neu; carcinoembryonic antigen, muc-1, and the like.

Tumors of interest for imaging include carcinomas, e.g. colon, prostate, breast, melanoma, ductal, endometrial, stomach, dysplastic oral mucosa, invasive oral cancer, non-small cell lung carcinoma, transitional and squamous cell urinary carcinoma, etc.; neurological malignancies, e.g. neuroblastoma, gliomas, etc.; hematological malignancies, e.g. childhood acute leukemia, non-Hodgkin's lymphomas, chronic lymphocytic leukemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

Cancers of particular interest include breast cancers, which are primarily adenocarcinoma subtypes. Ductal carcinoma in situ is the most common type of noninvasive breast cancer. In DCIS, the malignant cells have not metastasized through the walls of the ducts into the fatty tissue of the breast. Infiltrating (or invasive) ductal carcinoma (IDC) has metastasized through the wall of the duct and invaded the fatty tissue of the breast. Infiltrating (or invasive) lobular carcinoma (ILC) is similar to IDC, in that it has the potential metastasize elsewhere in the body. About 10% to 15% of invasive breast cancers are invasive lobular carcinomas.

For imaging of pathogens, markers of interest include cell surface proteins specific for the pathogenic organism. For example, to follow the progress of lengthy chemotherapy in treating infections of Pseudomonas or Mycobacteria species, antigens specific to these organisms may be used for imaging. Imaging agents derived from nucleic acids such as antisense DNAs and RNAs can also serve as markers.

Dynamic Contrast Imaging Agents: Dynamic contrast enhanced MRI following intravenous administration of small-molecular agents that distribute rapidly in the extracellular space (so-called non-specific or ECF agents) is used widely in clinical tumor evaluations. These imaging agents have an enhanced signal at the site of diseased tissue, where the distribution of the contrast agent correlates with the presence of one or more genes or proteins of interest, e.g. proteins altering microvascular permeability, etc. Examples of ECF MRI contrast agents include gadopentetate dimeglumine (trade name: Magnevist) (Berlex Laboratories); gadodiamide (trade name: Omniscan) (Nycomed Amersham, Inc.); and gadoteridol (trade name: ProHance) (Bracco, Inc.)

The degree of signal enhancement seen on T1-weighted images is dependent on a number of factors including tissue perfusion, capillary permeability to contrast agent, volume and environment of the extracellular leakage space, native T1-relaxation time of the tissue, contrast agent dose, imaging sequence and imaging parameters, also on machine scaling factors. Increased enhancement of tumors relative to surrounding tissue relates to increased tumor vascularity, where parameters of interest include microvascular volume (MVD) and the rate of diffusion of ECF agents into the interstitial space. For example, vascular permeability (measured as the rate constant K₂₁) is thought to be dependent on tissue VEGF expression in breast tumors.

Objective analysis of T1-weighted contrast-enhanced MR images is performed by either measuring signal intensity changes (semi-quantitative analysis) or by fitting pharmacokinetic models to the tissue contrast medium concentration-time curves (quantitative analysis). Semi-quantitative descriptors of contrast enhancement include onset time, initial and mean gradient of the upsweep of enhancement curves, maximum signal intensity and washout gradient. Signal intensity time curve shapes have been correlated with the type of breast tumor.

Quantitative analysis of dynamic contrast-enhanced MR images using pharmacokinetic models can provide estimates of physiological parameters such as the transfer constant K^{trans} (formally called permeability-surface area product per unit volume of tissue), the volume of extravascular extracellular space (EES) per unit volume of tissue (Vₑ) and the rate constant (kₑₚ also called K₂,). When the contrast agent concentration is measured accurately and the type, volume and method of administration are consistent, then pharmacokinetic parameters acquired serially in patients and between patients imaged at different scanning sites can be directly compared. Color-coded pixel maps of quantitative pharmacokinetic parameters can also be superimposed on the anatomical gray-scale images.

In the methods of the present invention, MRI data is correlated with indicators of disease, particularly angiogenesis of tumor tissues. The analysis of the expression patterns of genes that are involved is correlated to the MRI enhancement data. For example, comparisons are performed on the gene expression profiles of areas of the same tumor with different imaging characteristics. By carrying out this type of studies across a number of tumors, a cluster of genes is identified that is correlated to the imaging characteristics. Of particular interest is the correlation of genetic expression with tumor induced angiogenesis and response to anti-angiogenic therapies. Biopsy of areas with imaging parameters indicative of angiogenesis should lead to tissue analysis that will give targets related to the angiogenic process. Tissue samples obtained without image guidance would not be focused on those regions and miss the corresponding targets.

In one example, regions of increased contrast enhancement are found to correlate with changes in the mRNA of proteins strongly associated with the extracellular matrix. The matrix seems to contain factors to increase permeability to small molecules such as Gd(DTPA) and/or has properties to retain these molecules long enough to be observed using standard clinical imaging sequences. Also the mRNA of proteins associated with metastatic cell lines and tumors, e.g. MGP, PDGF receptor, etc., are up regulated in the contrast enhancing areas. Strong contrast-enhancing regions in tumors may be associated with changes in the extracellular matrix that creates an environment favorable to metastatic spread of the disease. Thus, the environment which can be assessed with image guided tissue analysis, in which the tumor cells are exposed, is as important as the cell's taxonomy when staging tumors in clinical disease and defining targets for therapeutic interventions.

Imaging Label. There are many different labels and methods of labeling known to those skilled in the art. Examples of the types of labels that can be used in the present invention include radioactive isotopes, paramagnetic isotopes, and compounds that can be imaged by positron emission tomography (PET). Those skilled in the art will know of other suitable labels for binding to the targeting molecules used in the invention, or will be able to ascertain such using routine experimentation. Furthermore, the binding of these labels to the targeting molecules can be done using standard techniques common to those skilled in the art.

Examples of metallic ions (radioisotopes) that can be bound to imaging agents of the instant invention are ^{99m}Tc, ¹²³I, ¹³¹I, ⁹⁷Ru, ⁶⁷Cu, ⁶⁷Ga, ¹²⁵I, ⁶⁸Ga, ⁷²As, ⁸⁹Zr, and ²⁰¹Tl. ^{99m}Tc and ¹¹¹In are preferred.

For diagnostic in vivo imaging, the type of detection instrument available is a major factor in selecting a given radionuclide. The radionuclide chosen must have a type of decay that is detectable by a given type of instrument. In general, any conventional method for visualizing diagnostic imaging can be utilized in accordance with this invention. Another important factor in selecting a radionuclide for in vivo diagnosis is that its half-life be long enough that it is still detectable at the time of maximum uptake by the target tissue, but short enough that deleterious radiation of the host is minimized. A currently used method for labeling with ^{99m}Tc is the reduction of pertechnetate ion in the presence of a chelating precursor to form the labile ^{99m}Tc-precursor complex, which, in turn, reacts with the metal binding group of a bifunctionally modified chemotactic peptide to form a ^{99m}Tc-chemotactic peptide conjugate.

Liposomal imaging agents: refers to particles having one or more encapsulating membranes formed by amphiphilic molecules (such as lipids for example) and in particular particles having a bilayer membrane and an enclosed aqueous core, which are carriers for the site specific delivery of diagnostic imaging agents. They can be used to selectively target specific organs, such as the liver, spleen, lung, lymphatic system, and bone marrow or they can be retained in the vasculature.

The composition and size of liposomal agents can be selected to control their biodistribution and, since one can use as the bulk of the membrane-forming molecules naturally occurring phospholipids, their metabolization and metabolite elimination can pose far fewer problems than is the case with macromolecular reagents.

Polymerized vesicles. One approach to imaging angiogenesis is to utilize the changes in vascular endothelium to target imaging agents, for example see Sipkins et al. (1998) Nat. Med. 4:623-626. Immunohistochemical staining of tumors shows co-localization of these polymerized vesicles in the tumor blood vessels without penetration into the surrounding tumor tissue.

Preferred carriers are lipid-based; biocompatible polymerized vesicles (PV) specifically engineered to be labeled with metals, and targeted to specific vascular endothelial cell surface receptors known to be upregulated during angiogenesis, e.g. endothelial cell receptors. To form antibody conjugated polymerized vesicles (AbPVs), a particle may be constructed containing biotinylated lipids. Via biotin molecules on the particle surface, an avidin bridge may be utilized to attach biotinylated antibodies. PVs containing 0.5% biotinylated lipid, 29.5% Gd+³ chelator lipid, 10% amine-terminated lipid and 60% filler lipid (PDA) may be formed by incubating with avidin and the biotinylated (VCAM) antibody. Alternatively, covalent attachment methods may be developed to conjugate the antibody to the polymerized vesicle.

Therapeutic conjugates: The binding moieties of the imaging agents may be further used to deliver a therapeutic agent, which agents may include chemotherapeutics, toxins, radiotherapeutics or radiosensitizing agents. Chemotherapeutic compounds, when contacted with and/or incorporated into a cell, produce an effect on the cell including causing the death of the cell, inhibiting cell division or inducing differentiation. Toxins are agents that produce the death of the cell on contact or introduction into the cell. Radiotherapeutic refers to radionuclides which when contacted with and/or incorporated into a cell, produce the death of the cell.

Analysis of expression. Various methods are used to determine the gene and protein expression profile from a patient sample, or from a region of a sample corresponding to an imaging feature of interest. Once the subset of genes or proteins of interest is identified, the data is used developing therapeutic and/or diagnostic targets, new imaging agents, selecting the most appropriate imaging or therapeutic cocktail for an individual, and the like. By analysis of gene expression in a specific region, or patient sample, the specific genes and proteins that are present in the cells of interest are determined. One or more imaging/therapeutic agents are then selected which have the best specificity for the individual patient, cell type, and disease.

Methods exist for obtaining information on gene expression using functional genomics and proteonomics in different tissues from individuals. Commercial systems (Affymetrix Inc., Sunnyvale, Calif. and Incyte Pharmaceutical, Santa Clara, Calif.) and research laboratories have generated profiles relating gene expression to clinical outcomes of individual patients. Microarrays can be purchased or made according to methods known in the art. Exemplary are PCT Application Serial No. WO95/35505, published Dec. 28, 1995; U.S. patent no. 5,445,934, issued Aug. 29, 1995; and Drmanac et al., Science 260:1649-1652. Quantitative monitoring of gene expression patterns with a complementary DNA microarray is described in Schena et al. (1995) Science 270:467. A DNA microarray system for analyzing complex DNA samples using two-color fluorescent probe hybridization is described in Shalon et al. (1996) Genome Res 6:639.

Microarray methodology is used to identify genes that are upregulated in the target tissue. By hybridization of the mRNA or a derivative thereof to probes of known sequence, the microarray is used to determine the set of genes that are expressed in the cells of interest, and particularly the subset of genes that is upregulated relative to surrounding or normal tissue.

RNA is isolated from cells of interest, which may be a biopsy sample taken from a patient, or an experimental sample, e.g. xenograft, tissue culture, etc. Various methods for RNA isolation are known in the art and may be found in standard reference works. In a preferred method a control preparation of RNA from normal cells is also prepared.

The RNA preparations may be directly labeled, or may be amplified by a number of techniques known in the art. For example, labeled cDNAs can be generated by the use of reverse transcriptase, where a different label may be used for the control and the test sequence. Alternatively, RNA can be amplified using a complementary primer linked to an RNA polymerase promoter region complement and then anti-sense RNA (aRNA) is transcribed from the cDNA by introducing an RNA polymerase capable of binding to the promoter region (see U.S. Pat. No. 5,891,636, Van Gelder et al.)

Suitable labels include nucleotides conjugated to fluorochromes, e.g. Cy2, Cy3, Cy5, fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N, N,N′,N′-tetramethyl-6-carboxyrhodamine (TAMRA)), that may be included in a standard amplification reaction containing appropriate primers. Unincorporated fluorescent nucleotides are removed (e.g. by size exclusion chromatography) prior to analysis. Preferably, where a control sample is included, the fluorescent reporter used to label the control sequences emits a fluorescent signal at an excitation and/or emission wavelength detectably distinct from that of the fluorescent reporter used to label the test sequence.

Hybridization of the labeled sequences to a microarray is accomplished according to methods well known in the art. In a preferred embodiment, the two probes are combined to provide for a competitive hybridization to a single microarray. Hybridization can be carried out under conditions varying in stringency, preferably under conditions of high stringency (e.g., 4× SSC, 10% SDS, 65° C.) to allow for hybridization of complementary sequences having extensive homology (e.g., having at least 85% sequence identity, preferably at least 90% sequence identity, more preferably having at least 95% sequence identity). Where the target sequences are native sequences the hybridization is preferably carried out under conditions that allow hybridization of only highly homologous sequences (e.g., at least 95% to 100% sequence identity).

Two-color fluorescent hybridization can be utilized to assay the representation of the test sequences in relation to the control sequence. From the ratio of one color to the other, for any particular array element, the relative abundance of that sequence in the two samples can be determined. In addition, comparison of the hybridization of the two sets of probes provides an internal control for the assay.

Microarrays can be scanned to detect hybridization of the selected and the unselected sequences using a custom built scanning laser microscope as described in Shalon et al., Genome Res. 6:639 (1996). A separate scan, using the appropriate excitation line, is performed for each of the two fluorophores used. The digital images generated from the scan are then combined for subsequent analysis. For any particular array element, the ratio of the fluorescent signal from the amplified selected cell population DNA associated is compared to the fluorescent signal from the unselected cell population DNA, and the relative abundance of that sequence in the selected and selected library determined.

Genes over represented in the pathologic cell population relative to a control tissue, e.g. normal cell populations, other regions of the diseased tissue, earlier or later time points in disease progression, etc., represent genes potentially suitable for use as targets of imaging agents.

Proteomics: In another embodiment of the invention, the tissue analysis is performed directly on the proteins present in the targeted tissue, e.g. by ELISA, two dimensional protein gel analysis, MALDI-TOF (matrix assisted laser deabsorption ionization—time of flight); etc.

In one application of the invention, the individualized analysis is performed by proteomics. A biological sample comprising the cells targeted for image analysis is subjected to two successive separation steps in order to identify suitable targets for imaging. In the first separation step, proteins are separated according to one physical or chemical property so as to generate a one-dimensional array, for example, separation by isoelectric focusing. The cells may be surface labeled to enhance visualization of proteins present in or on the cell.

In the second separation step, the proteins in the first array are separated according to a second physical or chemical characteristic so as to generate a two-dimensional array; for example, proteins separated by isoelectric focusing are subjected to SDS-PAGE perpendicular to the first axis. The separated biomolecules are stably maintained in the two-dimensional array for subsequent imaging.

The two-dimensional array is imaged with a detector to generate a computer-readable output that contains a set of x, y coordinates and a signal value for each detected protein. Computer-mediated analysis of the computer-readable output is performed, resulting in a computer-readable profile that represents the relative abundance of each protein on the array and its attributes as deduced from its x, y coordinates in the two-dimensional array.

The computer-readable profiles provide analysis to identify one or more proteins that satisfy the criteria of over-expression relative to surrounding tissue. In one embodiment, a first array is compared to a control array to identify proteins that are over-expressed in the diseased tissue.

Database of Imaging Agents: The cocktail of therapeutic or imaging agents used in the present method may be selected from a library, or database of such agents. The contents of such a database include publicly known and available imaging agents, e.g. gadopentetate dimeglumine, gadodiamide, and gadoteridol; agents having specificity for HER-2, integrins, prostate specific antigen, etc. The correlated gene or gene product of such agents, i.e. VEGF, an integrin protein, pathogen protein, etc. is represented either by its corresponding nucleic acid, or protein in a screening microarray or proteomics gel, as described above.

In one embodiment, the information obtained from analysis of biopsy and other pathologic tissue samples is used to refine and build a database of suitable imaging agents. For example, the identification of sequences over-represented in an mRNA sample from a tissue of interest is used to clone or characterize the corresponding genetic sequence, which information can then be used to develop specific binding moieties.

These may be employed for producing all or portions of the encoded proteins. For expression, an expression cassette may be employed, providing for a transcriptional and translational initiation region, which may be inducible or constitutive, where the coding region is under the transcriptional control of the transcriptional initiation region, and a transcriptional and translational termination region. Various transcriptional initiation regions may be employed which are functional in the expression host. The peptide may be expressed in prokaryotes or eukaryotes, usually prokaryotes, in accordance with conventional ways, depending upon the purpose for expression. For large-scale production of the protein, a unicellular organism, such as E. coli, B. subtilis, S. cerevisiae may be used as the expression host cells.

With the availability of the protein in large amounts, by employing an expression host, the protein may be isolated and purified in accordance with conventional ways. A lysate may be prepared of the expression host and the lysate purified using HPLC, exclusion chromatography, gel electrophoresis, affinity chromatography, or other purification technique. The purified protein will generally be at least about 80% pure, preferably at least about 90% pure, and may be up to and including 100% pure. Pure is intended to mean free of other proteins, as well as cellular debris.

The polypeptide is used for the production of antibodies, where short fragments provide for antibodies specific for the particular motif, and larger fragments or the entire protein allow for the production of antibodies over the surface of the polypeptide. Antibodies may be raised to the wild type or variant forms of the protein. Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988.

### METHODS

A patient lesion is imaged through any one of the methods described above. Regions and/or time points of interest for screening are detected by the imaging methods. Tissue samples may be excised, biopsied, and the like to provide samples for analysis, particularly samples having specific imaging features of interest. The tissue regions corresponding to the imaging features of interest are then further analyzed for the presence of variations in mRNA or protein expression, e.g. to correlate the expression patterns with the observed imaging variation. As described above, the analysis will utilize a high throughput system for expression analysis, including hybridization to polynucleotide microarrays, or proteomics. The cells of interest may be compared to cells from other regions of the lesion, to earlier time points in the disease, to normal tissues of similar type, and the like. The information from the high throughput analysis is used to determine the expression in the tissue region of interest, thereby providing molecular targets for imaging and therapy.

Differences in both spatial and temporal resolution over the tumor can be observed with imaging, and are found to correlate with changes in gene expression profiles. The targeted gene or gene product may then be used as a target for drug screening, for targeted therapeutic intervention, for further targeted imaging, e.g. to follow the course of disease through drug treatment, and the like. The selection of the molecular target is based on evaluation of factors that include specificity of expression, i.e. the expression on targeted tissue relative to surrounding tissue, the level of expression on the target tissue, the distribution of expression over the tissue, e.g. a tumor mass; the availability of specific binding reagents; etc.

Where it is desired to provide a patient specific imaging or therapeutic regimen, the individual sample is assessed for the optimal targets. For example, in patients with tumors expressing low level of alpha-v-beta-3 receptors but high levels of other integrins, the integrins that are most highly expressed relative to surrounding tissue is selected for imaging or therapy.

The selected imaging agents are administered to the patient in accordance with conventional practice to image the targeted tissue. Repeated imaging may be performed over time, to monitor the progression of disease, response to therapeutic agents, and the like.

The information about an individual patient from the gene expression analysis and from the imaging is also useful in therapeutic uses, where the specific binding moieties of the imaging agents are conjugated to therapeutic moieties, as described above. The therapy can be adjusted to utilize the optimal molecular target. This therapy can then be monitored with target-specific imaging method over time. In cases where different regions of the pathologic tissues have different patterns of target molecule upregulation, target-specific imaging can generate a spatial distribution map. As a result, combination image-guided therapeutic regimens can be developed.

Functional genomic and proteonomic analysis in tissue samples obtained with image-guidance is crucial for defining the target-specific imaging and image-guided therapeutic and/or diagnostic approaches. If needed, the therapy can be further adjusted by changing to other agents specific to different molecules that are upregulated and reassessed by imaging and functional and proteonomic analysis. In this way, the therapy is always adjusted and followed with imaging to correlate with the genetic changes in the pathologic tissues over space and time.

In cases where the pattern of gene expression changes significantly over time as indicated by target-specific imaging, a repeat tissue sample obtained with image guidance with functional genomics analysis may be required to guide further target-specific imaging and therapy.

In addition, there are imaging methods and therapeutic agents that are heavily dependent on vascular permeability for efficacy. Currently, it is difficult to determine in an individual whether the response to these types of diagnostic and therapeutic approaches will be favorable. Since vascular permeability is related to a host of different gene expression patterns (e.g. VEGF, VEGFr, FGF, etc.) functional genomics data can be used to guide the selection of diagnostic and therapeutic methods for individual patients. Imaging can then be used to follow the spatial and temporal distribution of the vascular permeability, for example using iron particles, liposome or polymer vesicles, or dynamic contrast enhanced MRI or CT.

There are also commercially available imaging and therapeutic agents that are designed for various receptors upregulated, or metabolic pathways affected, in different disease states. Information regarding the spatio-temporal distribution of these changes are crucial in designing the individualized diagnostic and treatment regimens for the patients.

For example, anti-CD20 antibody for treatment of B cell lymphoma has been used successfully in some patients. Similarly, anti-Her-2 antibody is only efficacious in the subpopulation of breast cancer patients expressing the receptor.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXAMPLE 1

### Correlation of MRI and cDNA Microarray Analysis of Breast Cancer

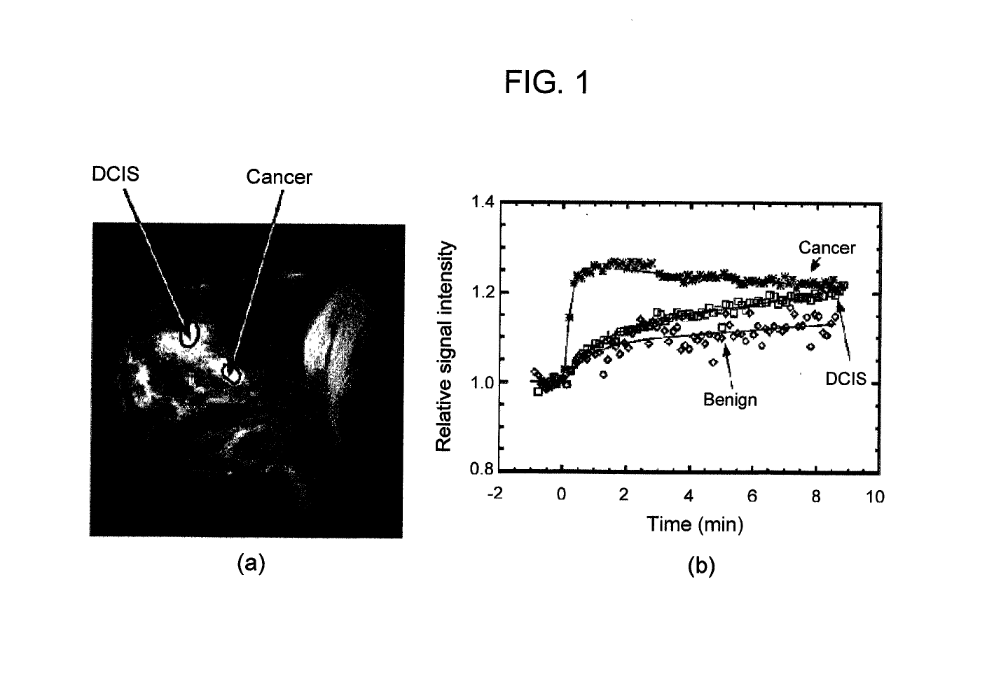

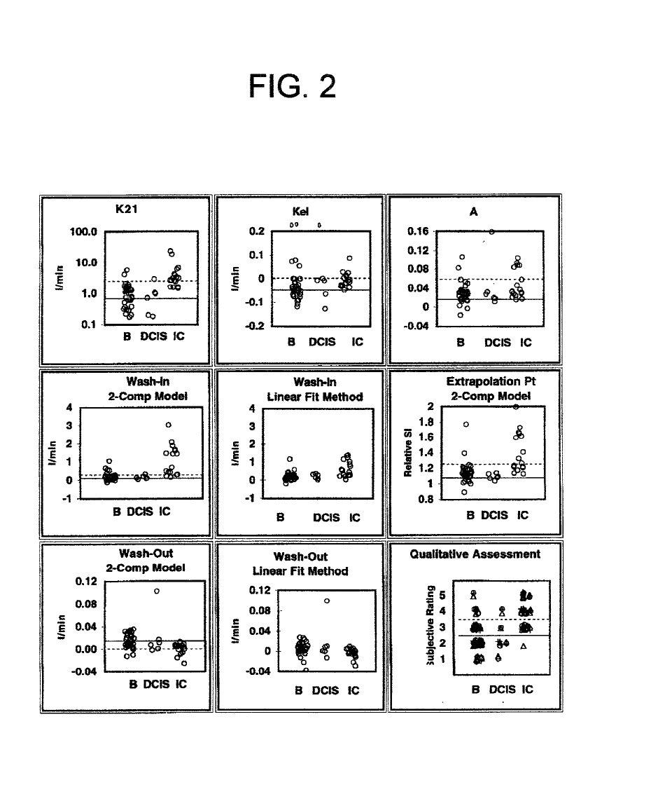

Dynamic spiral breast MR imaging was performed in 3 women scheduled for resection of invasive carcinoma of the breast. Gadolinium-enhanced images were obtained at 12 locations through the whole breast every 7.8 seconds for 8.5 minutes after bolus injection of contrast material. Once the lesion was identified on the spiral images, a ROI was drawn around the lesion and a curve of average signal intensity (SI) as a function of time was determined for that lesion using a workstation. ROIs were drawn to include as much as the lesion as possible without including any associated spiculation to minimize noise in the time-SI data. Thus, the SIs obtained represent an average for the entire lesion. Curves were then normalized to the SI of the nonenhanced lesion. The time-intensity curves from the ROIs were then analyzed by means of a two-compartment pharmacokinetic model were analyzed. High-resolution images and the time-intensity curves with the various parameters, as illustrated in FIG. 1. That the tumor in Patient #1 has a much lower average K₂₁ value and presumably a lower microvascular permeability as compared to Patient #2 and #3. Larger tumors in Patient #1 and Patient #3 have higher variability in the K₂₁ values over the tumor as compared to the smaller tumor in Patient #2. FIG. 2 demonstrates a time-intensity curves of the inner and peripheral regions of the large tumor in Patient #3. Notice the big difference in the two curves, highlighting the heterogeneity of the imaging characteristics over a single tumor.

All three patients underwent tumor resection and the flash-frozen breast tumor samples were collected. Total mRNA was extracted from each of the tumor specimens, and used to prepare cDNA labeled with Cy5 (red). As a common reference, to allow comparison of the mRNA levels for each gene among all the samples, a pool of mRNA from a diverse set of 11 cancer cell lines was used to prepare a cDNA probe labeled with Cy3 (green). This reference probe provided a “common denominator” for the fluorescence ratio measurements for each gene, for each tumor RNA sample, allowing all the measurements of expression of a given gene to be normalized to the same (arbitrary) scale.

Table 1 summarizes the expression data of a set of genes commonly associated with inflammation and a set of genes potentially associated with angiogenesis in the three patients. Notice that the tumor in Patient 1, which has the lowest K₂₁ value of the three tumors, also has the lowest expression level of all the listed genes that are commonly associated with inflammation. Notice that the expression levels of only some genes that are potentially associated with angiogenesis correlate with the K₂₁ levels. The expression levels of CD34 antigen, basic fibroblast growth factor (bFGF), and alpha V integrin are lower in Patient #1 as compared to Patients #2 and 3. However, the expression levels of the genes for fibronectin, vascular endothelial growth factors (VEGF) are not correlated to the K₂₁ levels. Notice that the expression levels in the two copies of VEGF-B are very similar in the different patients, again attesting to the robustness of our system.

Materials and Methods

Probe preparation and microarray hybridization. Total RNA is isolated from tissue samples by homogenization in TRIzol reagent (Gibco-BRL), followed by mRNA purification using a kit (Invitrogen). Using this protocol, one typically obtains 20 micrograms of mRNA per gram of tissue. When the mRNA yields are sufficient, Cy3 and Cy5-labelled dNTPs were used for reverse transcription to prepare labeled cDNA copies directly from total mRNA isolated from each tumor or tissue sample. Typically between 1 and 2 micrograms of mRNA is used for each hybridization. The microarray hybridizations and washes are carried out using published procedures. When the sample sizes or mRNA yields are insufficient to allow adequate labeling using a standard procedure it is intended to use an amplification procedure, closely following the Eberwine procedure, which produces amplifications of 200 fold with very little alteration in our measured expression ratios, when the same procedure is applied to both RNA samples compared in the differential hybridizations (correlation coefficients between complete sets of expression ratios measured with and without amplification typically exceed 0.9, when 10 ng of mRNA is used for amplification).

Common reference mRNA mixture for comparative hybridizations. To allow all measurements of gene expression made in separate microarray hybridizations to be comparable on the same quantitative scale, each hybridization will involve comparison of mRNA from a specific tumor with a common reference mRNA sample used in every microarray hybridization in this study. The reference RNA has no physiological significance in its own right, but is intended solely to provide a detectable hybridization signal at virtually every spot in the microarrays. Thus, it is prepared by mixing RNA's from a very diverse set of cell lines, that can be grown in arbitrarily large quantities under highly reproducible tissue culture conditions. In the present experiments, the reference probe is composed of equal quantities of mRNA from the following cell lines: MCF7, Hs578T, NTERA2, Colo205, OVCAR-3, UACC-2, MOLT4, RPMI-8226, NB4 (+retinoic acid), SW872, and HepG2.

MR imaging protocol. All breast MR images will be obtained on 1.5-T imagers (Signa LX; GE Medical Systems, Milwaukee, Wis.) with use of a dedicated four-coil phased array breast coil (MRI Devices, Waukesha, Wis.). The imaging protocol will include preliminary axial T1-weighted spin-echo imaging (repetition time msec/echo time msec=300/17; matrix 512×192; section thickness, 5 mm; field of view 36-44 cm; one signal acquisition) to assess axillary lymph nodes. Single-breast sagittal MR imaging will then be performed, including the dynamic two-dimensional multisection spiral MR examination. This is a multisection spiral, k-space-trajectory, gradient-echo sequence. A water-selective spectral-spatial excitation is used to prevent signal from fat and obviates subtraction methods and their associated problems during data analysis. With use of eight spiral interleaves, 12 sections can be obtained through the whole breast within 7.68 seconds at a resolution of 1.1 mm (960/6.8; filed of view 20 cm; matrix 188×188, flip angle 90 degrees, one signal acquisition). Section thickness depends on breast size and will range from 7 to 10 mm. Images were reconstructed off line on a dedicated workstation by using a gridding algorithm that includes zero-order motion correction and linear off-resonance correction, yielding 66 images over 8 minutes 26 seconds at each section location. Before the examination is started, an antecubital intravenous catheter will be placed in the arm opposite the breast being imaged. An intravenous bolus (0.1 mmol per kilogram of body weight) of gadolinium contrast material (Magnevist, Berlex Laboratories, Wayne, N.J.) will be administered by means of power injector (rate 3 musec; total injection time, 5-8 seconds; 20-mL saline solution flush immediately after injection) 35 seconds after dynamic spiral MR imaging is started. Thus, allowing for a typical circulation time for contrast material to reach the breast, approximately 46 seconds of baseline nonenhanced dynamic data will be obtained, followed by 7 minutes 30 seconds of dynamic contrast material-enhanced data.

### EXAMPLE 2

### Endothelial-Targeted Imaging of Angiogenic Vessels

Targets identified by imaging, for example integrins, find use in imaging and therapy. Other receptors identified by with image guided tissue analysis can be conjugated to a particle or particles for imaging and therapy, where particles may include liposomes, polymers and the like.

PV Synthesis and Characterization. To form antibody conjugated paramagnetic polymerized vesicles (AbPVs), we constructed a particle containing biotinylated lipids. Via biotin molecules on the particle surface, an avidin bridge was utilized to attach biotinylated antibodies. PVs containing 0.5% biotinylated lipid, 29.5% Gd+³ chelator lipid, 10% amine-terminated lipid and 60% filler lipid (PDA) were formed by incubating with avidin and the biotinylated anti-vascular cell adhesion molecule (VCAM) antibody in a ratio of 2.7 to 1.

Ab-PV Enhanced MRI of Tumor-Induced Angiogenesis in Rabbits. V2 carcinoma cells were inoculated into the thigh muscle or placed subcutaneously in New Zealand white rabbits. The rabbits were closely monitored until a palpable tumor was established. For in vivo MR studies, rabbits with palpable tumors (approximately 1-3 cm in diameter) were injected intravenously with either 5 ml/kg anti-αᵥβ₃ (LM609)-labeled AbPVs (1 mg antibody/kg, 0.005 mmol Gd⁺³/kg) or control AbPVs with isotype matched control antibodies MR imaging was performed using a 1.5 T GE Signa MR imager using an extremity coil and the following imaging parameters: TR=300 ms, TE=18 ms, NEX=2, FOV=16 cm, 256×256 matrix, slice thickness=3 mm. MR images were obtained immediately prior to contrast injection and at immediate, 30 minutes, 1 hour and 24 hours post-contrast injection in the coronal plane. The rabbits were euthanized immediately following the last MR imaging experiment and the tumor tissues were harvested for immunohistochemical studies. The MR findings of a V2 carcinoma carrying rabbit injected with LM609-labelled AbPVs showed no immediate, 30 minutes and 1 hour post-contrast injection enhancement of the tumor or tumor margin occurs as compared to the pre-contrast image, whereas at 24 hours post-contrast injection, enhancement of the tumor margin was clearly visible. Isotype matched controls showed low contrast enhancement in 24 hour post-contrast injection in both tumor models.

These results also show that the zone of αᵥβ₃ upregulation seen on immunohistochemistry corresponds very well to the zone of enhancement seen on LM609-labelled AbPVs enhanced MRI. The stain is localized to the vessels and not distributed throughout the tumor cells

Ab-PV gamma imaging of Tumor-induced Angiogenesis in rabbits. We have also performed imaging and therapy of a solid tumor with LM609-AbPVs labeled with indium 111 (¹¹¹In). Targeting of LM609-AbPVs was demonstrated by parenteral injection in the Vx2 carcinoma rabbit model by scintographic imaging of the tumor with LM609-¹¹¹In AbPVs. Scintographic imaging of the Vx2 carcinoma implanted in the rabbit thigh was performed using 100 nm LM609-¹¹¹In PVs after a single IV injection. Prior to imaging, the tumor volume was approximately 3 cm³. Gamma emission was monitored over a 72 h period and showed accumulation of 22% of the total injected radiation in the tumor at 72 h relative to 3% for the ¹¹¹In-AbPVs, which lacks the anti-αᵥβ₃ integrin antibody LM-609. The accumulation of the targeted vesicle represents approximately 1% of the injected dose per gram of tumor tissue. However, this dose is a severe underestimation of the dose to the tumor vasculature because of the confinement of the vesicle to the tumor vasculature, which represents a small fraction of the total tumor mass. The blood-pool half-life was 18 h for the LM609-¹¹¹In AbPVs and was similar to the complex without antibody.

### EXAMPLE 3

### Conjugation of Imaging Agents to Polymerized Vesicles

In some aspects of the present invention, the imaging agents may be conjugated to activated polymerized vesicles (Targesome Inc., Palo, Alto, Calif.). Particles may be conjugated with metal chelators such as DTPA attached to the surface, using a simple combinatorial material synthesis approach followed by rapid screening for polymerized vesicles (PVs) that efficiently bind both radionuclides for scintigraphy and gadolinium for MR imaging. The chelators may be attached to the surface with metal addition to a preformed particle; or the chelators with metal attached to the monomers prior to particle construction.

PVs with the desired metal affinity will be conjugated to targeting agents such as monoclonal antibodies to confer the specificity for cell receptors of interest, for example using two commonly used radionuclides, technetium-99m and indium-111 in order to ultimately image and quantify receptor binding events in vivo. Identified PV formulations with the desired properties are conjugated with commercially available monoclonal antibodies specific PECAM-1 (CD-31, the integrin αᵥ (CD51), VCAM-1 (CD54) and VEGF receptor (flk-1). Peptide mimetics based on the RGD motif are also used as model for small molecule targeting groups towards the integrins. The resulting ligand and antibody-targeted radionuclide-bound PVs are tested in vitro using purified human PECAM-1 (CD-31), the integrin αᵥ (CD51), VCAM-1 (CD54) and VEGF receptor (flk-1) in an enzyme-linked immunosorbant assay (ELISA).

Coupling of Monoclonal Antibody and Ligands to the Nanoparticle Surface. Efficient radionuclide binding formulations are selected for covalent coupling of a monoclonal antibody PECAM-1 (CD-31), the integrin αᵥ (CD51), VCAM-1 (CD54) and VEGF receptor (flk-1) to the nanoparticle's surface. Covalent coupling methods are preferred over forming biotin-avidin conjugates because of considerations in developing these materials for clinical applications. These murine monoclonal antibody are all commercially available (Chemicon International, Inc., Temecula, Calif.). Coupling methods include: 1) EDAC coupling to a carboxylate surface; 2) Michael addition of free thiols on the surface of the antibody to maleamides coupled to the surface of the polymerized vesicle; 3) formation of a thiourea by coupling the free lysines of the antibody to an isothiocyanate surface; 4) ion-exchange of the antibody on to surfaces with high surface potential energies. The peptide GRGDC (Bachem, Torrence, Calif.) will also be coupled to the PVs using the maleamide surface and the free thiol present on the cysteine. After the coupling of the monoclonal antibody and ligands and subsequent purification of the conjugated PVs by gel exclusion chromatography, the resulting complex will be assayed in vitro for binding using an ELISA with commercially available purified receptors. After the complex is characterized, it will be tested again for Tc-99m, In-111 and gadolinium binding. Based on our earlier studies, we expect to use approximately 0.1 to 1 mg of targeted nanoparticle constructs are needed for animal imaging studies using a murine tumor model.

Anti-angiogenesis therapies. Angiostain, endostatin and thalidomide are currently undergoing human trial as anti-angiogenesis therapies and their use in pre-clinical animal models is well documented. These materials are commercially available and antibodies toward these molecules have also been developed and are commercially available (Chemicon Temecula, Calif.)

Previous investigations utilizing vascular targeting agents and tumor therapy have include peptides derived from in-vivo screening of phage libraries directed at various targets including aminopeptidase N, apoptosis effectors, matrix metalloproteases, and integrins. Another strategy was the thrombosis of tumor vessels as a result of delivery of a truncated form of tissue factor to the vessels using a biispecific antibody. Vascular targeted radioimmunotherapy targets (RIT) include endoglin, and thrombomodulin. A Y(90) labeled anti-integrin antibody is used for vascular targeted radio-immunotherapy to evaluate the effects of radiation on targeted imaging for angiogenesis and apoptosis and its correlation with changes in gene profiling.

### EXAMPLE 4

### Image Guided Functional Genomics

To gain a better understanding of gene expression patterns in tumors, we have used contrast-enhanced magnetic resonance imaging (MRI) to non-invasively characterize regions within the same tumor in order to provide a correlate for genomic analysis. We compared the gene expression profiles of samples from a mouse tumor model obtained from contrast-enhancing and non-enhancing regions within the same tumor using MRI and functional genomics. We have analyzed 11,000 genes from these samples and have found that ten genes are upregulated in the contrast-enhancing areas, and one gene is upregulated in the non-enhancing region. Several of these genes encode extracellular matrix proteins. This study demonstrates that MRI can serve as a powerful, non-invasive tool for characterizing different regions of tumors to guide genomic analysis with both high spatial and temporal resolution.

The squamous cell carcinoma VII (SCC VII) is a transplantable, tumor that arose spontaneously in the abdominal wall of C3H/Km mice. It has subsequently been adapted for in vitro growth. The SCCVII tumor was initiated in fifteen C3H/Km mice at 10-12 weeks of age and 25-30 grams in body weight. For tumor implantation, 2×10⁵ SCCVII cells in 0.05 ml Hank's solution was injected subcutaneously in the left flanks of mice. Imaging of the tumors was started when they reached an approximate diameter ranging form 15-20 mm. At approximately 20 days, four tumors showed significant contrast enhancement with easy accessibility from both enhancing and non-enhancing regions to obtain enough tissue for microarray processing. These tumors were then used for image-guided genomic analysis.

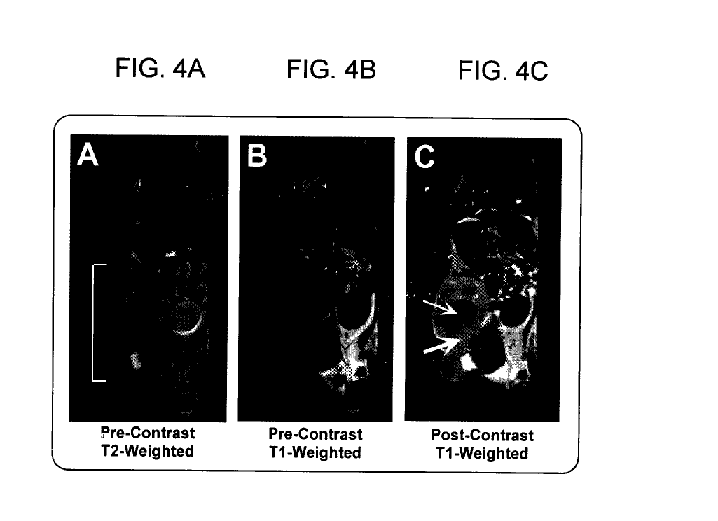

As outlined in FIG. 4 for tumor A, the area of high contrast enhancement on the T1-weighted image (Image C, thick arrow) and low contrast enhancement (thin arrow) were spatially marked and the tissue removed. The total RNA was isolated using TRIzol Reagents (GibcoBRL Life Technologies, Rockville, Md.) following procedures accompanying the reagent. The mRNA was then isolated using Fast Track® 2.0 Kit Protocol (Invitrogen, Carlsbab, Calif.). Affymetrix procedures were followed in obtaining the resulting biotin-labeled cRNA probes that were hybridized to the Affymetrix oligonucleotide microarrays (Mu11subA and Mu11subB, total of 11,000 genes). GeneChip Expression Analysis algorithms developed by Affymetrix were used to analyze data generated from the oligonucleotide microarrays (Affymetrix GeneChip® Expression Analysis Technical Manual, Chapter 2 and Appendix 5, 1999).

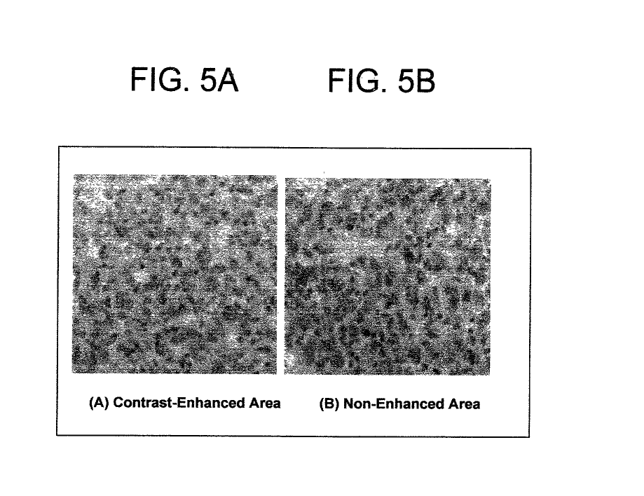

In order to see if any microscopic differences could be observed between the tissue samples, both the contrast enhancing and the non-enhancing regions were evaluated histologically. Using hematoxylin and eosin staining (H&E) one observes no difference between the enhancing and the non-enhancing regions of the tumor. In addition, no necrosis is observed at this stage of tumor growth and no significant inflammatory infiltrates are observed (FIG. 5).

The results of the gene array analysis are summarized in the Table 2. The table shows that significant spatial differences in gene expression profiles occur within the same tumor, and these differences are discernable using contrast-enhanced MRI. Significant up-regulation (average changes>2.0 fold) was observed in nine genes (Table 2).

TABLE 2
Gene expression profile in the contrast-enhanced (E) and non-enhanced (NE) regions of four SCC VII mouse tumors
(A-D). The table shows the gene name followed by its accession number (Acc. #).
The effective intensity values observed on the Affymetrix GeneChip ® for the non-enhanced (NE) and
contrast-enhanced (E) regions are shown followed by the fold change (FC) for the 4 tumors (A-D). The last
two columns represent the average fold change (Avg. FC) for the 4 tumors and its standard deviation (Std Dev FC).
The intensity value of the non-enhancing (NE) region is used as the baseline for calculating the FC in gene
expression for each tumor. The FC calculations include a series of statistical parameters considering background
and noise intensity within each gene chip (Affymetrix GeneChip ® Expression Analysis Technical
Manual Appendix 5, 1999, Affymetrix, Inc.). The up-and down-regulated genes are those with the avg. FC
of >2. ¹ The values in parenthesis represent very low expression.

| Tumor | | A | | | B | | | C | | | D | | | Avg. | Std |
|---|---|---|---|---|---|---|---|---|---|---|---|---|---|---|---|
| Gene Name | Acc. # | NE | E | FC | NE | E | FC | NE | E | FC | NE | E | FC | FC | Dev FC |

Up regulated

| | | | | | | | | | | | | | | | |
|---|---|---|---|---|---|---|---|---|---|---|---|---|---|---|---|
| matrix | D00613 | 938 | 2153 | 2.3 | 1842 | 3512 | 2 | 1242 | 2370 | 1.9 | 760 | 2436 | 3.2 | 2.35 | 0.59 |
| Gla protein | | | | | | | | | | | | | | | |
| fisp-12 protein | m70642 | 46 | 80 | 1.7 | 38 | 187 | 4.2 | 22 | 80 | 3.7 | 20 | 113 | 4.6 | 3.55 | 1.29 |
| CTLA-2-α | X15591 | 403 | 832 | 2.1 | 548 | 1298 | 2.5 | 528 | 1170 | 2.2 | 542 | 1181 | 2.2 | 2.25 | 0.17 |
| (cysteine | | | | | | | | | | | | | | | |
| protease) | | | | | | | | | | | | | | | |
| ECM | u64827 | 159 | 294 | 1.9 | 304 | 468 | 1.6 | 135 | 437 | 2.9 | 122 | 344 | 2.5 | 2.23 | 0.59 |
| associated | | | | | | | | | | | | | | | |
| protein SC1 | | | | | | | | | | | | | | | |
| IGFBP-3 | X81581 | 276 | 770 | 2.8 | 511 | 1416 | 2.8 | 282 | 619 | 2.5 | 325 | 1072 | 3.4 | 2.88 | 0.38 |
| apolipoprotein | AA036067 | 1519 | 4000 | 2.4 | 2800 | 4155 | 1.4 | 1444 | 3786 | 2.7 | 1264 | 3696 | 3 | 2.38 | 0.69 |
| E precursor | | | | | | | | | | | | | | | |
| alpha-globin | v00714 | 1521 | 6043 | 3.8 | 1866 | 5893 | 3 | 1298 | 4542 | 3.5 | 1070 | 3816 | 3.6 | 3.48 | 0.34 |
| beta-globin | X14061 | 1001 | 3614 | 3.6 | 1073 | 3886 | 3.6 | 609 | 2913 | 4.8 | 511 | 2327 | 4.6 | 4.15 | 0.64 |
| complex | | | | | | | | | | | | | | | |
| beta-1-globin | v00722 | 619 | 2422 | 3.9 | 788 | 2548 | 3.4 | 461 | 1791 | 4 | 439 | 1779 | 4.1 | 3.85 | 0.31 |
| PDGF receptor | X04367 | 199 | 465 | 2 | 346 | 453 | 1.7 | 334 | 437 | 1.3 | 230 | 474 | 2.1 | 1.78 | 0.36 |
| (precursor)¹ | | | | | | | | | | | | | | | |
| Down | | | | | | | | | | | | | | | |
| regulated | | | | | | | | | | | | | | | |
| mast cell | J05177 | 74 | 20 | −4.4 | 83 | 25 | −3.8 | 59 | (51) | −1.3 | 142 | 37 | −4.2 | −3.43 | 1.44 |
| protease-12 | | | | | | | | | | | | | | | |
| No change | | | | | | | | | | | | | | | |
| VEGF | W50462 | (7) | 28 | ˜1.7 | (4) | (3) | ˜−1.1 | (2) | (10) | ˜1.3 | (4) | (12) | ˜1.4 | 0.83 | 1.29 |
| precursor | | | | | | | | | | | | | | | |
| fibronectin | M18194 | 3726 | 3525 | 1 | 4018 | 4260 | 1.1 | 3369 | 3699 | 1.1 | 3444 | 3805 | 1.1 | 1.08 | 0.05 |

¹The average fold change (Avg. FC) of all the upregulated genes are greater than 2 except for PDGF receptor.

Six out of these nine proteins showing elevated mRNA levels in the contrast enhancing regions are associated with the extracellular matrix (ECM). The matrix Gla protein (MGP) is an extracellular protein associated with bone, dentin, and cartilage, and is dependent on vitamin-K γ-carboxylation. MGP is related to a class of proteins in the clotting cascade that get γ-carboxylated on glutamic acid residues using glutamic acid γ-carboxylase. It is the only protein with this property in which the function is not yet determined. It should be noted that the γ-carboxylation is inhibited by coumarin-type drugs and clinical studies have investigated the use of vitamin-K dependent γ-carboxylase inhibitors as anti-metastatic agents. The matrix Gla protein was also recently reported by Clark et al. to be associated with the process of metastasis in both mouse tumors and human melanoma tumors implanted in mice using genomic analysis. In addition, recent studies using serial analysis of gene expression (SAGE) to measure the mRNA levels of individual endothelial cells have identified MGP as an extracellular matrix endothelial associated protein.

In our studies the mRNA for MGP is upregulated in areas of high contrast enhancement using MRI in an unprecedented relationship to these other studies. This is the first indication that major differences in genomics occur within the same tumor and that these changes, indicated by radiographic changes, are associated with the ECM and may be related to metastatic potential of the tumor. MLP can therefore serve as a new molecular target for inhibiting metastatic cancers. This data demonstrates the power of performing image guided tissue analysis.

Other extracellular matrix proteins found highly up regulated were fisp-12, a gene that belongs to a family of proteins that respond to growth factors related to connective tissue. Fisp-12 is a cysteine-rich 35 kD secreted ECM protein that has a high (94% amino acid) identity with human connective tissue growth factor, a protein that binds to the platelet-derived growth factor (PDGF) receptor. We also found the mRNA of mouse CTLA-2-α, a homologue of the proregion of several cysteine protease precursors, highly up regulated in the contrast-enhancing region. Two other proteins associated with the extracellular matrix that also have consistently high mRNA levels in the contrast enhancing areas are ECM associated protein Sc1 and insulin-like growth factor binding protein 3 (IGFBP-3). ECM associated protein Sc1 is abundantly expressed in high endothelial venules, but has no defined function. IGFBP-3 modulates the bioavailability of IGF by sequestering this protein from circulation, thus effecting cell growth.

Interestingly we also see an increase in apolipoprotein E (Apo E) in the contrast enhancing areas of the tumor. Apo E is thought to be synthesized and secreted by astrocytes and has been of great interest as a participant in the pathogenesis of Alzheimer's disease. This gene is expressed in a variety of tumors but its role in tumor biology is undefined.

Another interesting set of proteins upregulated in the contrasting enhancing regions are the alpha and beta globins, the precursors used to form the oxygen carrying hemoglobin. Gene expression of these proteins involves an inter-play of several different mechanisms and may not be related to hemoglobin or myoglobin synthesis but related to a yet unrecognized mechanism involved in the contrast enhancing areas of the tumor.

The only major growth factor mRNA upregulated in the contrast enhancing areas was the PDGF receptor (i.e. the mRNA for the precursor to the cell surface receptor pre-PDGF). This gene has an average fold increase of approximately 1.8. However, the mRNA is upregulated in the enhancing region in all 4 tumors and has a fold change greater than two, in two of the tumors (Table 2, A and D). PDGF is a major mitogen that stimulates cell proliferation for connective tissue cells. Cells responsive to PDGF are dependent for their growth on contacts with matrix molecules mediated by integrins. Both PDGF and its receptors have been shown to have an angiogenic effect. It is proposed that overproduction of PDGF may be involved in autocrine and paracrine growth stimulation of human tumors, and activation of the receptors may be of critical importance in tumor progression.

The mRNA level most highly upregulated in the non-enhancing region compared to the enhancing area was the mast cell protease 12. These proteins are known to degrade the extracellular matrix and its presence may be related to altering the matrix in the non-enhancing region to increase perfusion in that area of the tumor.

Also of interest are genes that are not upregulated in the contrast enhancing areas or in any region of the tumor. Growth factors such as vascular endothelial growth factor (VEGF) have been implicated as one of the primary causes of vascular permeability in contrast enhancing tumor regions and have been thought to be related to “tumor angiogenesis”. Neocapillaries, as opposed to normal capillary beds, have been proposed to over express specific cell markers such as integrins, VEGF, fibroblastic growth factor (FGF), insulin-like growth factor, PDGF, and KDR (Flk-1). The expression of these factors and receptors are regulated by as of yet not fully understood mechanisms and pathways. Indeed the expression of VEGF in tissues causes MRI contrast enhancement using both small molecules such as gadopentate dimeglumine Gd(DTPA) and macromolecular agents such as albumin-Gd(DTPA). In our studies we observed no increase in VEGF precursor, which gives arise to VEGF1 and VEGF2. In fact, the mRNA levels of the VEGF precursor as compared to controls (i.e. mismatched base pair sets) were not detectable. VEGF either is unrelated to contrast enhancement at this stage of growth, or upregulated earlier in the tumor growth and vascularization.

The mRNA associated with fibronectin also shows no significant difference between the contrast enhancing and non-enhancing regions. In contrast to VEGF, however, the levels of fibronectin are high in both areas. Fibronectin is an extracellular matrix protein functioning as a ligand for the integrin family, and it mediates cell adhesion. Evidence from both genomic analysis and immunohistology has shown a correlation between fibronectin and tumorigenesis and metastasis. Our studies, however, indicate that at this stage of tumor growth, fibronectin is uniformly distributed over the tumors and is not related to contrast enhancement.

Contrast enhanced MRI can serve as a powerful tool for characterizing different regions of tumors for genomic analysis. We have observed that differences in both spatial and temporal resolution over the tumor using MRI correlate to changes in gene expression profiles. Regions of increased contrast enhancement best correlate with changes in the mRNA of proteins strongly associated with the extracellular matrix. Thus, the matrix seems to contain factors to increase permeability to small molecules such as Gd(DTPA) and/or has properties to retain these molecules long enough to be observed using standard clinical imaging sequences. Also the mRNA of proteins associated with metastatic cell lines and tumors such as MGP and PDGF receptor are up regulated in the contrast enhancing areas. These data show that strong contrast-enhancing regions in tumors are associated with changes in the extracellular matrix (the “milieu”) that creates an environment favorable to metastatic spread of the disease. This “milieu” is probably a combination of proteins from both the tumor cells themselves and the cells associated with the extracellular matrix. Thus, the environment in which the tumor cells are exposed to may be as important as the cell's taxonomy when staging tumors in clinical disease.

New imaging techniques can provide further discrimination of tissue samples for genomic analysis. With dynamic contrast enhanced MRI, the kinetics of contrast enhancement can be studied to provide more parameters for characterizing tissues in vivo. Similarly, MRI using tissue specific contrast agents and molecular imaging techniques can provide even more information about the tissue of interest. In addition, clinical imaging modalities such as computed x-ray tomography (CT), positron emission tomography (PET), single photon emission computed tomography (SPECT) can provide new means to correlate imaging with genomic analysis. Also optical imaging methods combined with laser-capture micro-dissection can provide further discrimination of samples obtained from image-guided biopsies down to the cellular level.

Contrast enhanced MRI can serve as a powerful noninvasive tool for characterizing different regions of tumors and provide correlates for genomic analysis with both high spatial and temporal resolution. This information is valuable in discovering new therapeutic and diagnostic aspects.

### EXAMPLE 5

### Molecular Targets Detected by Temporal Changes in Magnetic Resonance Imaging and Genomic Analysis

Tumors at different stages of growth can have distinct contrast-enhanced magnetic resonance imaging (MRI) characteristics. For example, tumors can exhibit similar MR T1-weighted (T1-wt) images while their T2-weighted (T2-wt) images differ significantly. Oligonucleotide microarrays were used to investigate the mechanistic basis for such MRI differences of MRI contrast enhancing (E) and non-enhancing (NE) tissue using murine cancer models. Differential genomic profiles were compared to the temporal stages of tumor growth to screen for potential molecular targets.

C3H/Km mice at ages of 10-12 weeks were transplanted with murine squamous cell carcinoma VII cells. For each experiment, 2×10⁵ cells in 0.05 ml Hanks' solution were implanted subcutaneously into the left flanks of mice. Tumors were imaged when they reached a diameter of ˜10-20 mm. A clinical 3 Tesla MR Scanner was used with a custom designed quadrature coil. Standard clinical spin echo (T1-wt) and fast spin echo (T2-wt) scan protocols were used. Post-contrast T1-wt image was obtained after the injection of a gadolinium-DTPA via tail vein. E and NE regions were spatially marked and the tumor tissue surgically harvested.

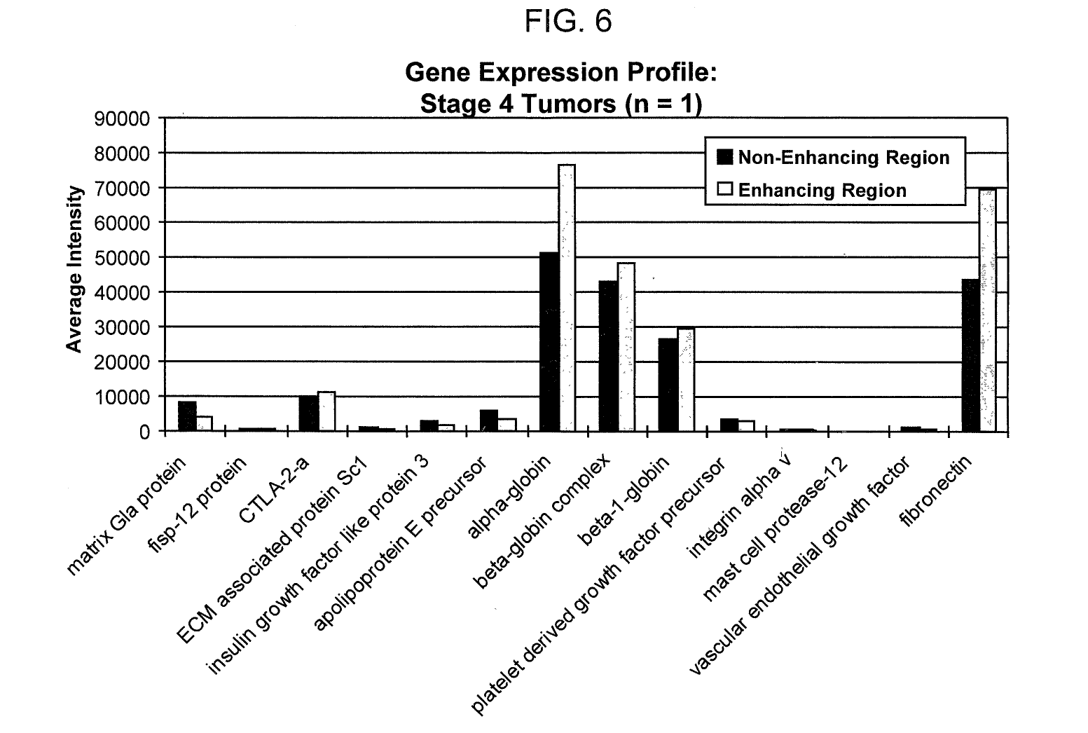

Pre-contrast T2-wt images of 2-week-old implanted tumors have no significant enhancement, indicating a solid mass with no significant necrosis. However, the pre-contrast T2-wt images of the three-week-old tumors show differential enhancement patterns, suggesting fluid accumulation due to the onset of necrosis. Both two and three-week-old tumors show E and NE regions in the post-contrast T1-wt images. Total RNA was extracted from tissue sampled from these E and NE areas for microarray hybridization analysis. We observed gross differences in gene expression profiles between the 2 and 3-week-old tumors. Significant up-regulation (mRNA levels>2 fold change) was observed in 7 genes from the 2-week-old non-necrotic tumors; none of these seven genes was up-regulated in the 3-week-old tumor. See FIG. 6. Individual gene expression patterns of 3-week-old tumors were highly variable but appear to be related to 5 classes of proteins.

In these experiments we have observed distinct gene expression profiles associated with different stages of tumor growth that exhibit different T2-wt images but similar post-contrast T1-wt images. From this image-guided genomics screen, we have identified several gene products that are candidates as molecular targets for cancer diagnosis and therapeutics.

### EXAMPLE 6

### Image Guided Proteomics in Human Glioblastoma Multiforme: New Clinical Technique for Molecular Target Discovery

Magnetic resonance image (MRI) guided surface enhanced laser desorption ionization mass spectroscopy (SELDI-MS) and MALDI-TOF was used to define differences in protein expression patterns between contrast-enhanced (CE) and non-enhanced (NE) regions of human glioblastoma multiforme (GBM). GBM has a classic appearance on MRI as an expansile mass with central necrosis, ring enhancement, and a large surrounding region of white matter edema. These patterns reflect the tumor's pathologic pattern of necrosis, hemorrhage and neovascularity. Standard neuropathological examination, radiographic examination, as well as surgical examination are useful in classification and staging, but are limited in terms of defining tumor borders and efficacious treatment options. Genomic studies have also shown both intra- and inter-tumor heterogeneity, making sampling of tissue for further microarray analysis a critical parameter to determine molecular targets in human GBM. Image guided proteomics is herein shown to provide a means to elucidate mechanisms responsible for the imaging patterns and pathologic variability seen in GBM and to offer a novel technique for discerning new molecular targets.

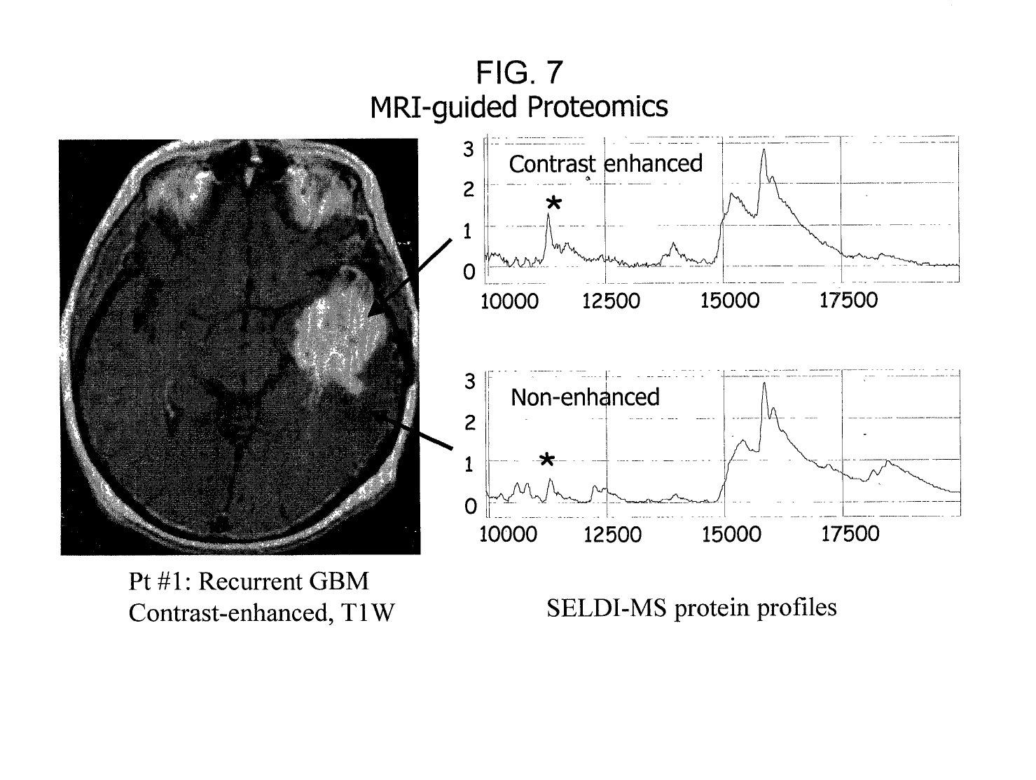

Total protein or membrane-associated protein was extracted from snap-frozen, freshly resected tumor samples guided by MRI characteristics. These extracts were then applied to cationic and hydrophobic surfaces for SELDI-MS and MALDI-TOF. The results of this analysis revealed several spatial differences in protein expression patterns between CE and NE regions. The most significant difference in all patients studied was the appearance of a ˜10 kDa protein in the CE region of the tumor but not in the NE region. In an effort to identify this protein, we performed RT-PCR for extracellular matrix proteins identified in our previous studies on mouse tumor models. See FIG. 7. Among these was a ˜10 kDa gene product that matched our SELDI-MS results. This gene product was shown to be matrix-Gla protein that has been previously reported to be associated with tumor metastatic potential. Matrix-Gla protein can therefore serve as a new molecular target for GBM.

This study represents an example of MRI-guided proteomics in human tumors and establishes that there are spatial differences in protein expression patterns within tumors that occur synchronously with MRI contrast differences. This spatial variability can account for the wide heterogeneity seen in previous pathological and genetic reports of this tumor. Combining sophisticated imaging tools such as MRI, characteristic image appearance, selective tumor sampling based on the images, and new proteomics technology, specific patterns of protein expression can be identified in GBM. The ability to identify spatial tumor-specific proteins correlated with imaging characteristics also provides exciting opportunities in the design of improved diagnostic and therapeutic agents.

All publications mentioned herein are incorporated herein by reference for the purpose of describing and disclosing, for example, the compounds and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

TABLE I

| | | |
|---|---|---|
| Patent #1 | Patent #2 | Patent #3 |

MR parameter

| | | | |
|---|---|---|---|
| K21 | 0.02830 | 0.04091 | 0.04370 |
| Genes associated with inflammation | | | |
| Intercellular adhesion molecule 2 | 1.32 | 1.87 | 1.29 |
| Intercellular adhesion molecule 3 | 0.22 | 0.58 | 0.44 |
| Platelet/endothelial cell adhesion | 4.21 | 8.50 | 11.99 |
| molecule | | | |
| Selectin E (endothelial adhesion | 1.50 | 1.55 | 2.99 |
| molecule 1) | | | |
| Vascular cell adhesion molecule 1 | 1.61 | 3.61 | 3.52 |
| Genes potentially associated | | | |
| with angiogenesis | | | |
| CD34 antigen | 10.14 | 20.38 | 19.91 |
| Fibronectin | 0.59 | 0.53 | 0.48 |
| Fibroblast growth factor 2 (basic) | 1.17 | 2.12 | 1.77 |
| Integrin, alpha V (vitronectin | 0.56 | 1.66 | 1.13 |
| receptor, alpha) | | | |
| Integrin, beta 3 | N/A | 0.25 | 0.52 |
| Vascular endothelial growth factor | N/A | 0.78 | 0.90 |
| Vascular endothelial growth factor B | 2.15 | 2.22 | 1.39 |
| Vascular endothelial growth factor B | N/A | 2.05 | 1.50 |
| (2nd copy) | | | |
| Vascular endothelial growth factor C | 0.67 | 0.77 | 0.93 |
| Vitronectin | 0.35 | 0.08 | 0.19 |

What is claimed is:

1. A method of screening for the discovery of disease associated molecular targets for diagnostic or therapeutic intervention, the method comprising;
performing in vivo imaging of diseased tissue to provide one or more in vivo images;
evaluating said in vivo image for imaging features;
obtaining a cellular sample from said diseased tissue, which sample corresponds to said imaging feature;
determining the expression of genes or gene products in said cellular sample;
comparing said expression in said cellular sample with a control tissue;
wherein genes or gene products upregulated in said cellular sample represent molecular targets for therapeutic or diagnostic intervention.

2. The method according to claim 1, wherein said in vivo imaging comprises magnetic resonance imaging (MRI).

3. The method according to claim 2, wherein said MRI is dynamic contrast MRI.

4. The method according to claim 1, wherein said step of determining expression of genes comprises hybridization analysis of probes derived from mRNA present in said cellular sample.

5. The method according to claim 1, wherein said step of determining expression of gene products comprises proteomic analysis.

6. The method according to claim 1, wherein said control sample comprises cells from said diseased tissue, but spatially or temporally separated from said cellular sample.

7. The method according to claim 1, wherein said in vivo imaging is selected from the group consisting of MRI, MRS, nuclear scintigraphy, PET, CT, ultrasonography, optical imaging, infrared imaging, and x-ray radiography.

8. A method for individualized imaging of a patient tissue, the method comprising:
analyzing a biopsy or excision sample from said patient for patterns of gene and protein expression;
determining from said patterns of gene expression, molecules that are over-expressed or under-expressed in said biopsy or excision sample, relative to normal or surrounding tissue;
selecting one or more imaging agents or methods, wherein the enhancement of signal obtained with said imaging agent correlates with the expression of one or more genes of interest in the target tissue;
preparing an image of said patient tissue utilizing said imaging agents.

9. The method of claim 8, wherein said imaging agent comprises a binding moiety specific for a cell surface molecule that is over-expressed in said target tissue; and an image enhancing label.

10. The method of claim 9, wherein said imaging agent is a small molecular weight, extracellular fluid space (ECF) MRI contrast agent.

11. The method according to claim 10, wherein said ECF agent is selected from the group consisting of: gadopentetate dimeglumine, gadodiamide, and gadoteridol.

12. The method according to claim 9, wherein said step of preparing an image is repeated throughout the course of a disease.

13. The method according to claim 9, wherein said analyzing a biopsy or excision sample for patterns of gene expression comprises hybridization of nucleic acids derived from said biopsy sample to a polynucleotide microarray.

14. The method according to claim 9, wherein said analyzing a biopsy or excision sample for patterns of gene expression comprises proteomic analysis of proteins present in said sample.

15. The method according to claim 9, wherein said patient tissue is a tumor tissue.

16. The method according to claim 15, wherein said tumor is a breast cancer.

17. The method according to claim 9, wherein said over-expressed molecules are proteins.

18. The method according to claim 17, wherein said proteins are adhesion molecules.

19. The method according to claim 18, wherein said adhesion molecules are integrins.

20. The method according to claim 9, wherein two or more binding moieties are selected for imaging.

21. The method according to claim 9, further comprising the step of treating said patient with a therapeutic moiety conjugated to said binding moiety.

22. The method according to claim 8, wherein said imaging agent is activated by an enzyme that is expressed in said target tissue and which can induce image enhancement by catalysis.
